(19) United States
(12) Patent Application Publication
(10) Pub. No.: US 2018/0050048 A1
(43) Pub. Date: Feb. 22, 2018

(54) CONJUGATE COMPOUNDS OF URSODEOXYCHOLIC, BERBERINE OR L-CARNITINE, AND COMPOSITIONS AND METHODS THEREOF

(71) Applicant: SHENZHEN HJGHTIDE-BIOPHARMAGEUTiCAL, LTD., Shenzhen (CN)

(72) Inventor: Liping LIU, Manassas, VA (US)

(21) Appl. No.: 15/557,199

(22) PCT Filed: Apr. 5, 2016
(86) PCT No.: PCT/CN2016/078414

§ 371 Date: Sep. 11, 2017

Related U.S. Application Data
(60) Provisional application No. 62/143,734, filed on Apr. 6, 2015.

(51) Int. Cl. A61K 31/575; A61K 31/202; A61K 31/192; A61K 31/385; A61K 31/56; A61K 31/194; A61K 31/216; A61K 31/225; A61K 31/198; A61K 31/191; A61K 31/375; A61K 31/203; A61K 31/455; A61K 31/4188; A61K 31/4375; A61K 31/58; A61K 31/197; A61K 31/155; A61K 45/06

(52) U.S. Cl. CPC A61K 31/575; A61K 45/06; A61K 31/192; A61K 31/385; A61K 31/56; A61K 31/194; A61K 31/216; A61K 31/225; A61K 31/198; A61K 31/191; A61K 31/375; A61K 31/203; A61K 31/455; A61K 31/4188; A61K 31/4375; A61K 31/58; A61K 31/197; A61K 31/155; A61K 31/202

(57) ABSTRACT

The invention provides novel conjugate compounds having at least one of a moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, and a moiety derived from berberine or L-carnitine or metformin or unsaturated fatty acid, or a derivative or analog thereof. The invention also relates to pharmaceutical compositions, methods of preparation and use of these conjugates in treating and/or preventing, for example, liver diseases or disorders, various diabetes, diabetic complications, dyslipidemia, obesity, metabolic syndromes, pre-diabetes, muscle atrophy, inflammation, and cancers. The compounds of this invention are also useful in improving liver functions in chronic viral associated liver diseases and alcohol-related liver diseases.

### PRIORITY CLAIMS AND RELATED APPLICATIONS

This application claims the benefit of priority from U.S. Provisional Application No. 62/143,734 filed Apr. 6, 2015, and the entire content of which is incorporated herein by reference in its entirety.

### TECHNICAL FIELD OF THE INVENTION

The invention generally relates to novel therapeutic compounds, pharmaceutical compositions, and methods of preparation and therapeutic use thereof. More particularly, the invention relates to novel conjugate compounds having at least one of a moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, and a moiety derived from berberine or L-carnitine or metformin, or a derivative or analog thereof. The invention also relates to pharmaceutical compositions, methods of preparation and use of these conjugates in treating and/or preventing, for example, liver diseases or disorders, various diabetes, diabetic complications, dyslipidemia, obesity, metabolic syndromes, pre-diabetes, muscle atrophy, inflammation, and cancers. The compounds of this invention are also useful in improving liver functions in chronic viral associated liver diseases and alcohol-related liver diseases.

### BACKGROUND OF THE INVENTION

Diabetes mellitus is a disorder of metabolism. It has become pandemic with an estimate of over 300 million people worldwide living with diabetes today. Without effective prevention, this number will grow to 500 million by 2030. There are three main types of diabetes: type 1 diabetes, type 2 diabetes, and gestational diabetes. Among them, type 2 diabetes, is the most common form of diabetes accounting for 90-95% of cases. Type 2 diabetes is characterized by impaired insulin secretion, increases hepatic glucose production, and decreased response of peripheral tissues to insulin, i.e., insulin resistance. Many therapeutic treatments are available for the management of type 2 diabetes, but they are often accompanied by various side effects. An optimal therapy should be safe and include early initiation of combination drugs with complimentary mechanisms of action.

Despite great efforts have been made in the understanding and management of diabetes, people with diabetes continue to face an increased risk of developing a number of serious complications including infliction of heart and blood vessels, eyes, kidneys, and nerves due to high blood glucose, high cholesterol, and high blood pressure. Cardiovascular disease is the most common cause of death in people with diabetes. Diabetic nephropathy caused by damage to small blood vessels in the kidney can lead to less efficient kidney or kidney failure altogether. Diabetic neuropathy is caused by damages to the nerves throughout the body when blood glucose and blood pressure are too high. Most people with diabetes will develop diabetic retinopathy causing reduced vision or blindness. Consistently high levels of blood glucose, together with high blood pressure and high cholesterol, are the main causes of diabetic retinopathy. Despite the great developments of anti-diabetic agents, there are significant unmet needs for therapeutics that can be used effectively for the treatment and management of diabetic complications.

Metabolic syndrome is the name for a group of risk factors that occur together (e.g., abdominal (central) obesity, elevated blood pressure, elevated fasting plasma glucose, high serum triglycerides, and low high-density cholesterol (HDL) levels). Metabolic syndrome has been demonstrated to increase the risk of developing cardiovascular disease, particularly heart failure, and diabetes. Studies have estimated the prevalence of metabolic syndrome in the U.S. to be around 34% in the adult population. While therapeutics are available, the first line treatment is change of lifestyle. High-dose statins, recommended to reduce cardiovascular risk, have been linked to higher progression to diabetes, especially in patients with metabolic syndrome.

Non-alcoholic fatty liver disease (NAFLD) is a form of fatty liver diseases that occurs when excessive fat is deposited in the liver. NAFLD is generally recognized to be associated with metabolic syndrome such as insulin resistance, hypertension and obesity. NAFLD affects about a third of the adult population in developed countries. Non-alcoholic steatohepatitis (NASH) is the most extreme form of NAFLD with chronic inflammation that can lead to progressive fibrosis (scarring), cirrhosis, and eventual liver failure and death. NASH resembles alcoholic liver disease, but occurs in people who drink little or no alcohol. A major feature of NASH is fat in the liver, along with inflammation and damage. Most people with NASH, an often “silent” liver disease, feel well and are not aware that they have a liver problem. Nevertheless, NASH can be severe and can lead to cirrhosis, when the liver is permanently damaged and scarred and no longer work properly.

Currently, there are no drugs approved for the treatment of NASH, which occurs in about a quarter of patients with NAFLD. The current standard of care for NASH involves weight loss and increased physical activities. NASH affects 2-5% of Americans and is becoming more common, possibly because of the greater number of Americans with obesity. In the past 10 years, the rate of obesity has doubled in adults and tripled in children.

Muscle atrophy is a decrease in the mass of the muscle, which can involve a partial or complete wasting away of muscle. Muscle atrophy occurs due to changes in the balance between protein synthesis and degradation. Muscular atrophy decreases qualities of life as the patient becomes unable to perform certain tasks or risks accidents (e.g., falling). Muscular atrophy is associated with aging and a serious consequence of different diseases, including cancer, AIDS, and diabetes. Comparing to non-diabetic older adults, elderly with type 2 diabetes have lower skeletal muscle strength, and is often associated with excessive loss of skeletal muscle mass. There are currently no drugs approved for the treatment of skeletal muscle atrophy.

Cancer is a group of diseases involving abnormal cell growth with the potential to invade or spread to other parts of the body. About 14 million new cases of cancer occurred globally in 2012. The most common types of cancer include lung cancer, prostate cancer, colorectal cancer and stomach cancer for men, and breast cancer, colorectal cancer, lung cancer, and cervical cancer for women. While many treatment options for cancer exist, including surgery, chemotherapy, radiation therapy, hormonal therapy, targeted therapy and palliative care, cancer remains a top health threat and is responsible for about 15% of all human deaths.

The therapeutics and methods currently available for the management of diseases or disorders such as liver diseases or disorders, diabetes, diabetic complications, dyslipidemia, obesity, metabolic syndromes, pre-diabetes, NAFLD, NASH, muscle atrophy, inflammation and cancers are suboptimal. There remains an ongoing and urgent need for novel and improved therapeutics and methods for such diseases or disorders.

### SUMMARY OF THE INVENTION

The invention is based in part on novel conjugate compounds. Each conjugate compound has at least one of a moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, and a moiety derived from berberine or L-carnitine or metformin, or a derivative or analog thereof. The invention is also based in part on pharmaceutical compositions and methods of preparation and therapeutic use of the conjugate compounds disclosed herein in treating and/or preventing various diseases and disorders, such as liver diseases or disorders, various diabetes, diabetic complications, dyslipidemia, obesity, metabolic syndromes, pre-diabetes, muscle atrophy, inflammation, and cancers. Liver diseases or disorders include non-alcoholic fatty liver disease, non-alcoholic steatohepatitis, cholestatic liver diseases or graft-versus-host disease of the liver. The conjugate compounds and pharmaceutical compositions thereof are also useful in improving liver functions in chronic viral associated liver diseases and alcohol-related liver diseases.

In one aspect, the invention generally relates to a compound having the formula of:

X—Y—Z (I)

wherein

(a) X is a moiety derived from a pharmacologically active organic base or acid;

(b) Z is a moiety derived from a pharmacologically active organic acid; and

(c) Y a covalent bond or a linker,

wherein at least

X is a moiety derived from berberine or a derivative or analog thereof, or L-carnitine or a derivative or analog thereof, or metformin or a derivative or analog thereof, or unsaturated fatty acid or a derivative or analog thereof,

Z is moiety derived from ursodeoxycholic acid or a derivative or analog thereof, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof.

In another aspect, the invention generally relates to a pharmaceutical composition comprising an amount of a compound having the formula of:

X—Y—Z (I)

wherein

(a) X is a moiety derived from a pharmacologically active organic base or acid;

(b) Z is a moiety derived from a pharmacologically active organic acid; and

(c) Y a covalent bond or a linker,

wherein at least

X is a moiety derived from berberine or a derivative or analog thereof, or L-carnitine or a derivative or analog thereof, or metformin or a derivative or analog thereof, or unsaturated fatty acid or a derivative or analog thereof,

Z is moiety derived from ursodeoxycholic acid or a derivative or analog thereof, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, wherein the amount is effective to treat, prevent, or reduce one or more diseases or disorders selected from liver diseases or disorders, diabetes, diabetic complications, pre-diabetes, dyslipidemia, obesity, metabolic syndromes, muscle atrophy, inflammation, and cancers or a related disease or disorder thereof in a mammal including a human, and a pharmaceutically acceptable excipient, carrier, or diluent.

In yet another aspect, the invention generally relates to a method for treating, reducing, or preventing a disease or disorder. The method includes: administering to a subject in need thereof a pharmaceutical composition comprising an amount of a compound having the formula of:

X—Y—Z (I)

wherein

(a) X is a moiety derived from a pharmacologically active organic base or acid;

(b) Z is a moiety derived from a pharmacologically active organic acid; and

(c) Y a covalent bond or a linker,

wherein at least

Z is moiety derived from ursodeoxycholic acid or a derivative or analog thereof, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, or

X is a moiety derived from berberine or a derivative or analog thereof, or L-carnitine or a derivative or analog thereof, or metformin or a derivative or analog thereof, or unsaturated fatty acid or a derivative or analog thereof, wherein the amount is effective to treat, prevent, or reduce one or more diseases or disorders selected from liver diseases or disorders, diabetes, diabetic complications, pre-diabetes, dyslipidemia, obesity, metabolic syndromes, muscle atrophy, inflammation, and cancers, or a related disease or disorder thereof in a mammal, including a human, and a pharmaceutically acceptable excipient, carrier, or diluent.

### Definitions

Unless defined otherwise, all technical and scientific terms used herein have the same meaning as commonly understood by one of ordinary skill in the art to which this invention belongs. General principles of organic chemistry, as well as specific functional moieties and reactivity, are described in “Organic Chemistry”, Thomas Sorrell, University Science Books, Sausalito: 2006.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Given the benefit of this disclosure, one of ordinary skill in the art will appreciate that synthetic methods, as described herein, may utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by preferably readily available, non-toxic reagents that do not attack the other functional groups; the protecting group forms an easily separable derivative or analog (more preferably without the generation of new stereogenic centers); and the protecting group has a minimum of additional functionality to avoid further sites of reaction. Oxygen, sulfur, nitrogen, and carbon protecting groups may be utilized. Examples of a variety of protecting groups can be found in Protective Groups in Organic Synthesis, Third Ed. Greene, T. W. and Wuts, P. G., Eds., John Wiley & Sons, New York: 1999.

It will be appreciated that the compounds, as described herein, may be substituted with any number of substituents or functional moieties. Throughout the specifications, groups and substituents thereof may be chosen to provide stable moieties and compounds.

As used herein, the term “effective” amount of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

As used herein, the term “treating, reducing, or preventing a disease or disorder” refers to ameliorating such a condition before or after it has occurred. As compared with an equivalent untreated control, such reduction or degree of prevention is at least 5%, 10%, 20%, 40%, 50%, 60%, 80%, 90%, 95%, or 100% as measured by any standard technique.

As used herein, the term “pharmaceutically acceptable excipient, carrier, or diluent” refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives and analogs, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the “an amount sufficient” refers to the amount of a compound, alone or in combination with another therapeutic regimen, required to treat, prevent, or reduce a metabolic disorder such as diabetes in a clinically relevant manner. A sufficient amount of an active compound used to practice the present invention for therapeutic treatment of conditions caused by or contributing to diabetes varies depending upon the manner of administration, the age, body weight, and general health of the mammal or patient. Ultimately, the prescribers will decide the appropriate amount and dosage regimen. Additionally, an effective amount may be an amount of compound in the combination of the invention that is safe and efficacious in the treatment of a patient having a metabolic disorder such as diabetes over each agent alone as determined and approved by a regulatory authority (such as the U.S. Food and Drug Administration).

As used herein, the “low dosage” refers to at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that reduces glucose levels and that is formulated for administration by inhalation will differ from a low dosage of the same agent formulated for oral administration.

As used herein, the “high dosage” is meant at least 5% (e.g., at least 10%, 20%, 50%, 100%, 200%, or even 300%) more than the highest standard recommended dosage of a particular compound for treatment of any human disease or condition.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. See, WO 2007/005643, WO 2007/005644, WO 2007/016361, and WO 2007/016431.

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Possible formulations include those suitable for oral, sublingual, buccal, parenteral (for example subcutaneous, intramuscular, or intravenous), rectal, topical including transdermal, intranasal and inhalation administration. Most suitable means of administration for a particular patient will depend on the nature and severity of the disease or condition being treated or the nature of the therapy being used and on the nature of the active compound.

### DETAILED DESCRIPTION OF THE INVENTION

The invention provides novel conjugate compounds, each of which has at least one of a moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof and a moiety derived from berberine or L-carnitine or metformin or unsaturated fatty acid, or a derivative or analog thereof. The invention also relates to pharmaceutical compositions, methods of preparation and use of these conjugates in treating and/or preventing liver diseases or disorders, various diabetes, diabetic complications, dyslipidemia, obesity, metabolic syndromes, pre-diabetes, muscle atrophy, inflammation, and cancers, for example. The conjugate compounds and pharmaceutical compositions of the invention are also useful in improving liver functions in chronic viral associated liver diseases and alcohol-related liver diseases.

A central feature of the invention is the unique and synergistic effect given rise by each of the two parts of the novel conjugate compound and the pharmaceutical composition thereof, i.e., a first pharmaceutically active portion and a second pharmaceutically active portion, that target a disease or disorder with complementary mechanisms of action thereby improving efficacy of treatment.

In one aspect, the invention generally relates to a compound having the formula of:

X—Y—Z (I)

wherein

(a) X is a moiety derived from a pharmacologically active organic base or acid;

(b) Z is a moiety derived from a pharmacologically active organic acid; and

(c) Y a covalent bond or a linker,

wherein at least

X is a moiety derived from berberine or a derivative or analog thereof, or L-carnitine or a derivative or analog thereof, or metformin or a derivative or analog thereof, or unsaturated fatty acid or a derivative or analog thereof,

Z is moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof.

In certain embodiments of the compound, Z is a moiety derived from ursodeoxycholic acid.

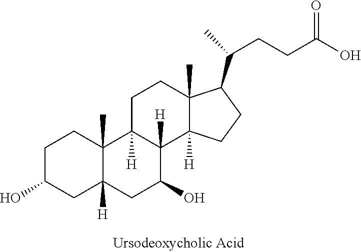

Ursodeoxycholic acid (UDCA or ursodiol, with the chemical names of 3α,7β-dihydroxy-5β-cholan-24-oic acid or (R)-4-((3R,5S,7S,8R,9S,10S,13R,14S,17R)-3,7-dihydroxy-10,13-dimethylhexadecahydro-1H-cyclopenta[a]phenanthren-17-yl)pentanoic acid) is a secondary bile acid, a substance naturally produced by the body that is stored in the gallbladder. Ursodiol is used to dissolve gallstones in patients as an alternative to surgery. Ursodiol is also used to prevent the formation of gallstones in overweight patients who are losing weight very quickly. Ursodiol works by decreasing the production of cholesterol and by dissolving the cholesterol in bile so that it cannot form stones. Ursodiol is also the first-line therapy for the treatment of PBC, PSC and cholestatic liver diseases. There have been limited studies of ursodiol on NASH, but the results were contradictory and inconclusive. Thus, the effect of ursodiol on NASH remains unclear.

In certain embodiments of the compound, Z is a moiety derived from a derivative or analog of ursodeoxycholic acid. Exemplary derivatives or analogs of ursodeoxycholic acid are listed in Table 1.

TABLE 1
Ursodeoxycholic acid Derivatives or Analogs

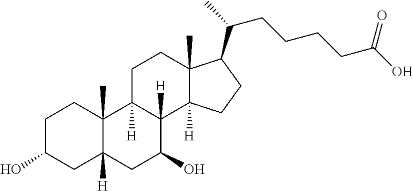

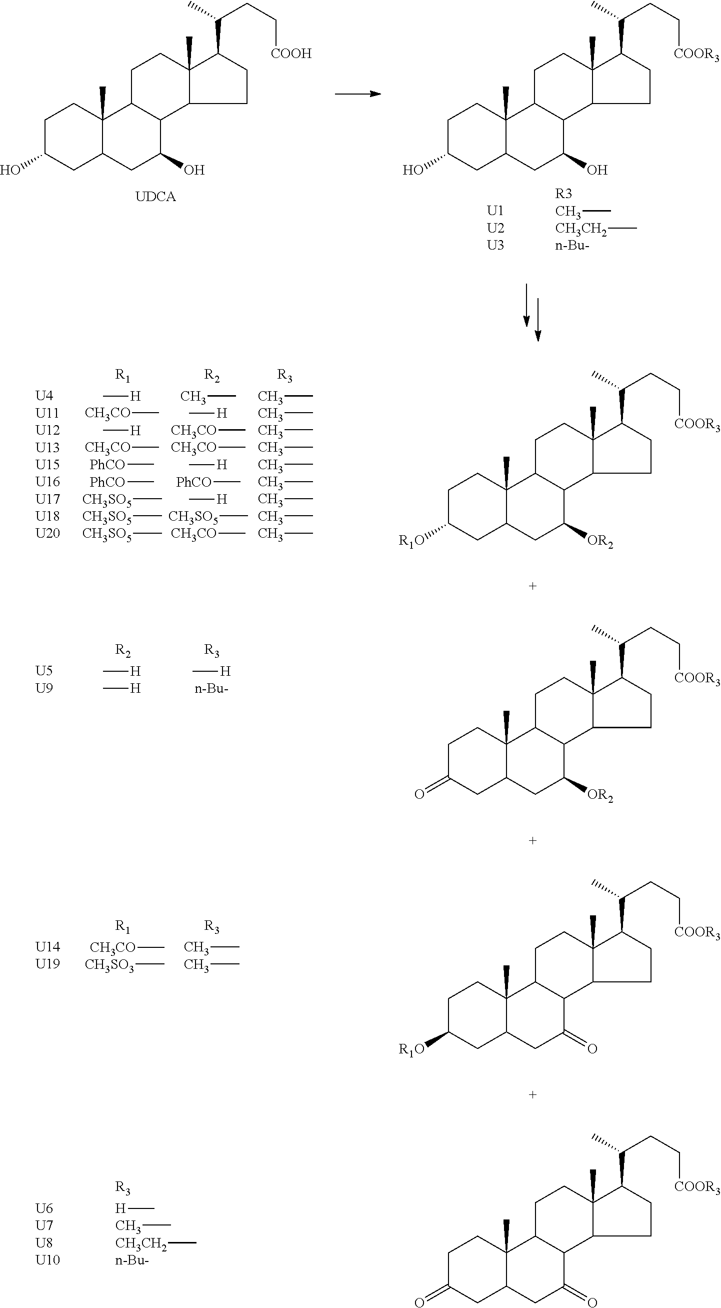

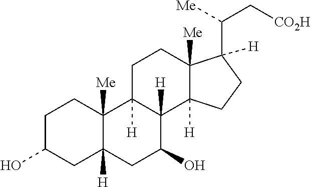

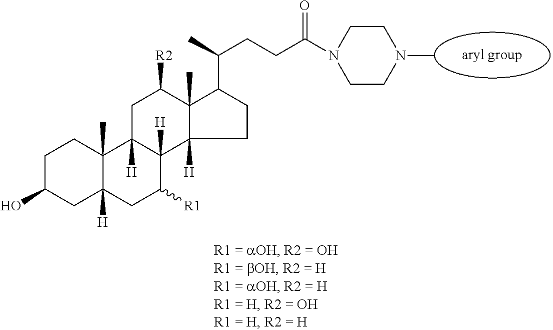

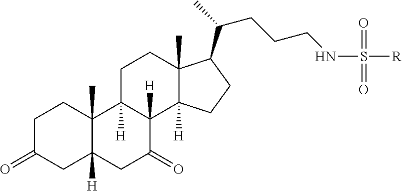

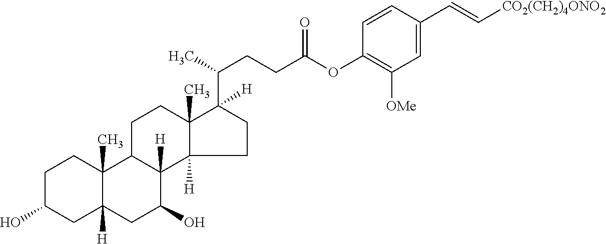

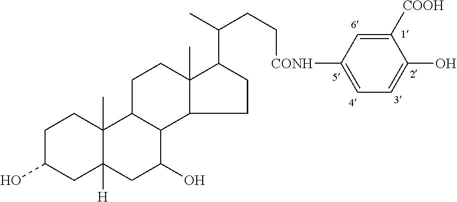

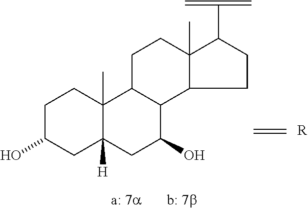

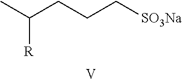

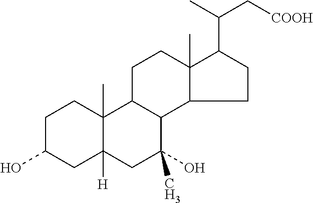 [SMILES: CC(CC(=O)O)C1CCC2C3C(CCC12C)C1(C)CC[C@@H](O)CC1C[C@@]3(C)O]

| | |
|---|---|
| 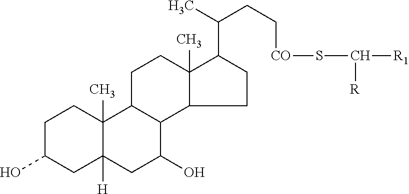 | (I) |

wherein R represents —H, —CH3 or —COCH and R represents —CONHCH₂COOH, —CH₂COOH or

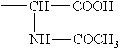

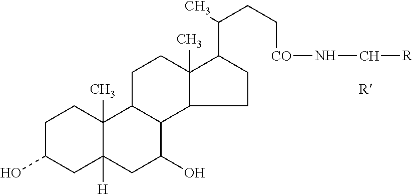

R is a radical selected from —CH2—SO₃H and —COOH and R′ is a radical selected from —H and
—(CH₂)₂—CONH, —CH₂—CONH₁, —(CH₂)₂—SCH₃, —CH₂—S—CH₂—COOH, respectively

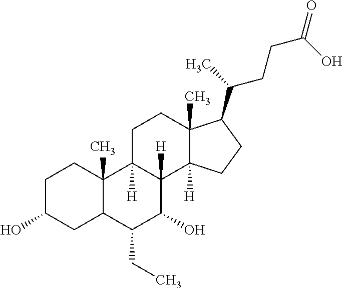

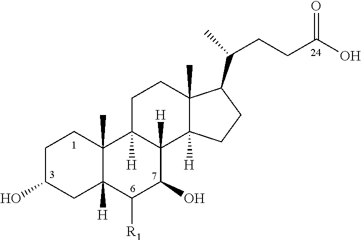

R₁ is selected from the group consisting of C₁-C₄ alkyl or a halogen; or an ester

In certain embodiments of the compound, Z is a moiety derived from eicosapentaenoic acid or docosahexaenoic acid.

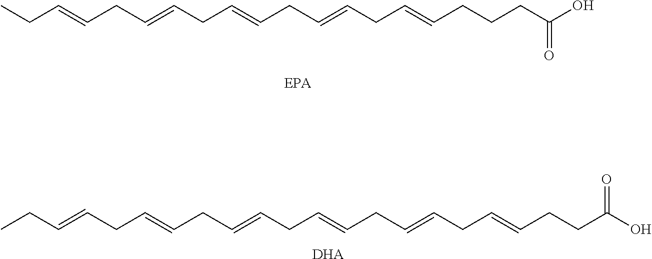

Eicosapentaenoic acid (EPA or (5Z,8Z,11Z,14Z,17Z)-5,8,11,14,17-icosapentaenoic acid), and docosahexaenoic acid (DHA, 4Z,7Z,10Z,13Z,16Z,19Z)-docosa-4,7,10,13,16,19-hexaenoic acid), are two best-investigated omega-3 polyunsaturated fatty acids. EPA is the active molecule in two FDA-approved anti-hypertriglyceridemic agents. It has been demonstrated that EPA and DHA can reduce free fatty acid and triglyceride synthesis and increase their disposal. Effects of EPA and DHA have also been demonstrated in reducing chronic inflammation, improving insulin resistance, maintaining heart and vascular health and reducing the risk of coronary heart disease.

In certain embodiments of the compound, Z is a moiety derived from rhein.

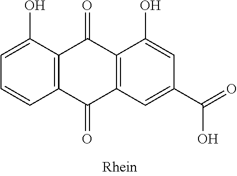 [SMILES: O=C(O)c1cc(O)c2c(c1)C(=O)c1cccc(O)c1C2=O]

Rhein (4,5-dihydroxy-9,10-dioxoanthracene-2-carboxylic acid), is one of the most important active components of rhubara (Rheum officinale), a traditional Chinese herb showing broad pharmacological effects. Rhein was reported to affect oxidative phosphorylation by inhibiting both electron transfer and ADP-driven H+ uptake in mitochondria, which is responsible for the formation of lipid peroxides. It has also demonstrated protective effects in diabetic nephropathy animal models in various studies. The pharmacokinetics of rhein have not been intensively studied in humans, an oral dose of 50 mg twice per day was shown to be safe when administered for five days to elderly patients with chronic congestive heart failure.

In certain embodiments of the compound, Z is a moiety derived from R-(+)-α-Lipoic acid.

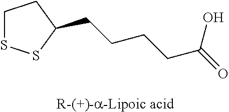 [SMILES: O=C(O)CCCC[C@@H]1CCSS1]

R-(+)-α-Lipoic acid ((R)-6,8-Dithiooctanoic acid, (R)-6,8-Thioctic acid, (R)-(+)-1,2-Dithiolane-3-pentanoic acid) was identified as a catalytic agent for oxidative decarboxylation of pyruvate and α-ketoglutarate. In human, R-(+)-α-lipoic acid exists in the body as a portion of several multi-enzyme complexes involved in energy formation and is an essential component of mitochondrial respiratory enzymes. R-(+)-α-Lipoic acid is best known for its potent anti-oxidant effects and has been used for the treatment of diabetic neuropathy, degenerative neuronal disease, atherosclerosis and other oxidative stress related abnormalities.

In certain embodiments of the compound, Z is a moiety derived from ursolic acid or corosolic acid.

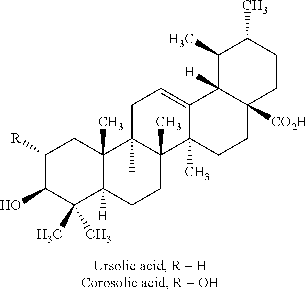

Ursolic acid ((1S,2R,4aS,6aR,6aS,6bR,8aR,10S,12aR,14bS)-10-hydroxy-1,2,6a,6b,9,9,12a-heptamethyl-2,3,4,5,6,6a,7,8,8a, 10,11,12,13,14b-tetradecahydro-1H-picene-4a-carboxylic acid) and corosolic acid ((1S,2R,4aS,6aR,6aS,6bR,8aR,10R,11R,12aR,14bS)-10,11-Dihydroxy-1,2,6a,6b,9,9,12a-heptamethyl-2,3,4,5,6,6a,7,8,8a,10, 11,12,13,14b-tetradecahydro-1H-picene-4a-carboxylic acid) are members of the pentacyclic triterpene acid class of compounds widely distributed in the plant kingdom. They have been shown to exhibit favorable pharmacological effects both in vivo and in vitro, including glucose reduction, anti-obesity, anti-inflammatory, reduce muscle atrophy, anti-cancer, liver protection, anti-oxidative stress.

In certain embodiments of the compound, Z is a moiety derived from hydroxycitric acid.

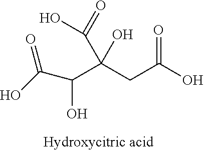 [SMILES: O=C(O)CC(O)(C(=O)O)C(O)C(=O)O]

Hydroxycitric acid (1,2-dihydroxypropane-1,2,3-tricarboxylic acid) is a derivative of citric acid found in a variety of tropical plants including Garcinia cambogia and Hibiscus subdariffa. Hydroxycitric acid is the active component of Garcinia cambogia extract, which has been widely utilized as dietary supplement for weight loss. There have been reports on hydroxycitric acid's effects in improving glucose tolerance, providing liver protection against toxicity associated with ethanol and dexamethasone, and controlling blood pressure. In addition, the compound has been found to reduce markers of inflammation in brain, intestines, kidney and serum.

In certain embodiments of the compound, Z is a moiety derived from a pharmacologically active organic acid, such as Cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid or biotin (Table 2).

TABLE 2
Exemplary Pharmacologically Active Organic Acids

| Name | IUPAC Name | Structure |
|---|---|---|
| Cinnamic acid | (E)-3-phenylprop-2-enoic acid | 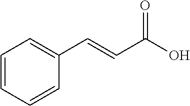 [SMILES: O=C(O)/C=C/c1ccccc1] |
| Cholic acid | (R)-4-((3R,5S,7R,8R,9S,10S, 12S,13R,14S,17R)-3,7,12- trihydroxy-10,13- dimethylhexadecahydro- 1H-cyclopenta[a] phenanthren- 17-yl)pentanoic acid | 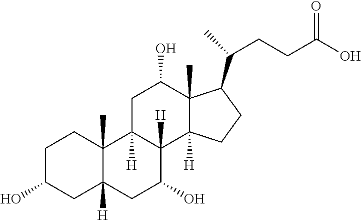 |
| Oleanolic acid | (4aS,6aR,6aS,6bR,8aR,10S, 12aR,14bS)-10-hydroxy- 2,2,6a,6b,9,9,12a-heptamethyl- 1,3,4,5,6,6a,7,8,8a,10,11,12, 13,14b-tetradecahydropicene- 4a-carboxylic acid | 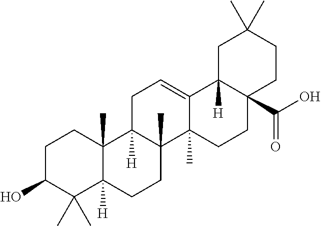 |
| Salicylic acid | 2-Hydroxybenzoic acid | 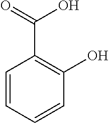 |
| Betulinic acid | (3β)-3-Hydroxy-lup- 20(29)-en-28-oic acid | 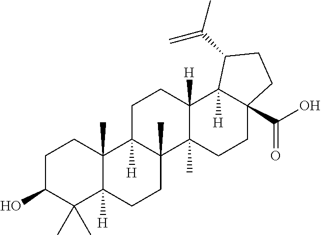 |
| Chlorogenic acid | (1S,3R,4R,5R)-3-{[(2Z)-3- (3,4-dihydroxyphenyl)prop-2- enoyl]oxy}-1,4,5- trihydroxycyclohexanecarboxylic acid | 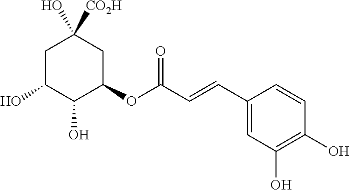 [SMILES: O=C(/C=C/c1ccc(O)c(O)c1)O[C@@H]1C[C@](O)(C(=O)O)C[C@@H](O)[C@H]1O] |
| Caffeic acid | 3-(3,4-Dihydroxyphenyl)- 2-propenoic acid 3,4- Dihydroxy-cinnamic acid trans-Caffeate 3,4- Dihydroxy-trans-cinnamate) (E)-3-(3,4-dihydroxyphenyl)- | 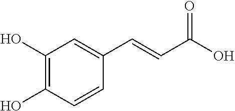 [SMILES: O=C(O)/C=C/c1ccc(O)c(O)c1] |
| | 2-propenoic acid 3,4- | |
| | Dihydroxybenzeneacrylicacid | |
| | 3-(3,4-Dihydroxyphenyl)-2- | |
| | propenoic acid | |
| Bassic acid | (4aR,6bS,9R,10R,11S,12aR, 14bS)-10,11-dihydroxy-9- (hydroxymethyl)-2,2,6b,9,12a- pentamethyl-1,2,3,4,4a,5,6,6a, 6b,7,9,10,11,12,12a,12b,13, 14b-octadecahydropicene-4a- carboxylic acid | 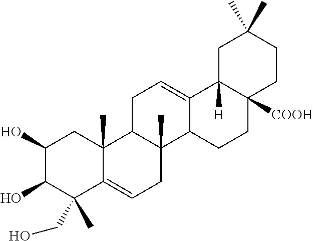 |
| Acetyl L- carnitine | (R)-3-Acetyloxy-4- trimethylammonio-butanoate | 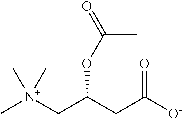 |
| S-allyl-L- cysteine sulphoxide | (2R)-2-amino-3-[(S)-prop- 2-enylsulfinyl]propanoic acid | 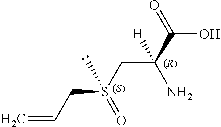 |
| S-methyl-L- cysteine sulfoxide | 3-(methylsulfinyl)-L-alanine | 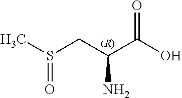 |
| Pantothenic acid | 3-[(2,4-Dihydroxy-3,3- dimethylbutanoyl)amino] propanoic acid | 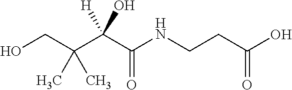 |
| Ascorbic acid | (5R)-[(1S)-1,2-dihydroxyethyl]- 3,4-dihydroxyfuran-2(5H)-one | 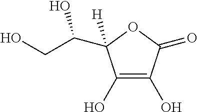 [SMILES: O=C1O[C@H]([C@@H](O)CO)C(O)=C1O] |
| Retinoic acid | (2E,4E,6E,8E)-3,7-dimethyl- 9-(2,6,6-trimethylcyclohexen- 1-yl)nona-2,4,6,8-tetraenoic acid | 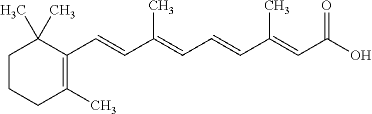 [SMILES: CC1=C(/C=C/C(C)=C/C=C/C(C)=C/C(=O)O)C(C)(C)CCC1] |
| Nicotinic acid | pyridine-3-carboxylic acid | 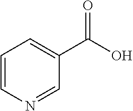 |
| Biotin | 5-[(3aS,4S,6aR)-2- oxohexahydro-1H-thieno [3,4-d]imidazol-4-yl] pentanoic acid | 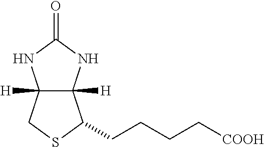 [SMILES: O=C(O)CCCC[C@@H]1SC[C@@H]2NC(=O)N[C@@H]21] |

In certain embodiments of the compound, X is a moiety derived from berberine.

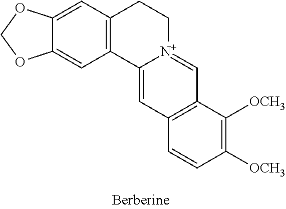 [SMILES: COc1ccc2cc3[n+](cc2c1OC)CCc1cc2c(cc1-3)OCO2]

Berberine (5,6-dihydro-9,10-dimethoxybenzo[g]-1,3-benzodioxolo[5,6-a]quinolizinium), an isoquinoline alkaloid isolated from Rhizoma Coptidis, has had a long history of medicinal use in China to treat various gastrointestinal diseases. Berberine is found in a variety of plants such as Berberis, Hydrastis canadensis, Xanthorhiza simplicissima, Phellodendron amurense, Coptis chinensis, Tinospora cordifolia, Argemone mexicana, and Eschscholzia californica. In the last two decades, in vitro and in vivo studies have demonstrated the efficacy of berberine when using alone or as a combination for diabetes, dyslipidemia, cancer, neuroprotection and cardiovascular diseases. Currently, berberine can be obtained commercially in the form of chloride, sulfate or tannate salt, with berberine hydrochloride being the form used in almost all previous studies. While some studies appear to show benefits of berberine in treating NAFLD, the low bioavailability and poor absorption of berberine in the current available forms, combining with the gastrointestinal side effects at high doses have made its clinical applications very challenging.

In certain embodiments of the compound, X is a moiety derived from a derivative or analog of berberine. Exemplary berberine derivatives or analogs are listed in Table 3.

TABLE 3
Berberine Derivatives or Analogs

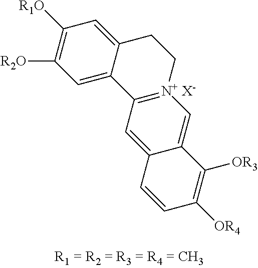

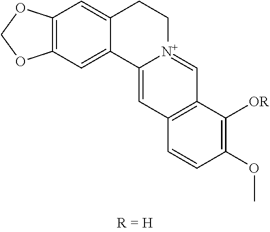

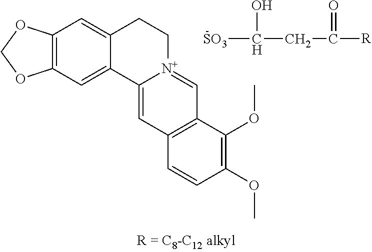

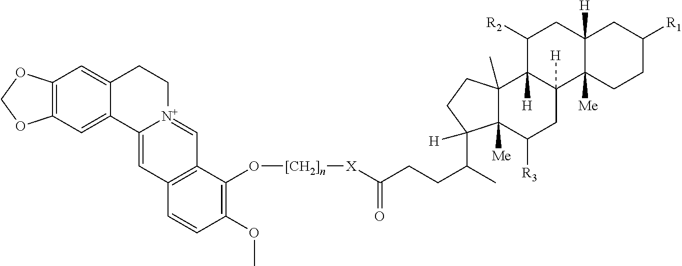

R₁ = OH, carbonyl; R₂, R₃ = H, carbonyl; n = 2-6; X = O
R₁ = OH, carbonyl; R₂, R₃ = H, OH, carbonyl; n = 2-6; X = NH

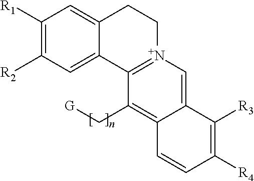

R₁, R₃, R₂, R₄ = OH, C₁-C₈ alkoxy, OCH₂O
G = Z—Ar, Y—Ar₂
Z = O(CH₂)ₘ, CONH(CH₂)ₘ, NHCO(CH₂)ₘ
Y = O(CH₂)ₘCH, CONH(CH₂)ₘCH, NHCO(CH₂)ₘCH
n = 1-5; m = 1-3; Ar = 5-15 membered unsaturated or aromatic ring

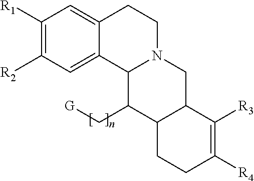

R₁, R₃, R₂, R₄ = OH, C₁-C₈ alkoxy, OCH₂O
G = Z—Ar, Y—Ar₂
Z = O(CH₂)ₘ, CONH(CH₂)ₘ, NHCO(CH₂)ₘ
Y = O(CH₂)ₘCH, CONH(CH₂)ₘCH, NHCO(CH₂)ₘCH
n = 1-5; m = 1-3; Ar = 5-15 membered unsaturated or aromatic ring

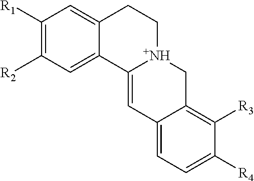

R₁, R₃, R₂, R₄ = OH, C₁-C₈ alkoxy, OCH₂O
G = Z—Ar, Y—Ar₂
Z = O(CH₂)ₘ, CONH(CH₂)ₘ, NHCO(CH₂)ₘ
Y = O(CH₂)ₘCH, CONH(CH₂)ₘCH, NHCO(CH₂)ₘCH
n = 1-5; m = 1-3; Ar = 5-15 membered unsaturated or aromatic ring

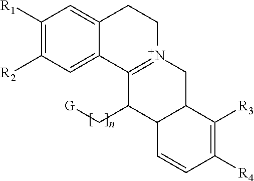

R₁, R₃, R₂, R₄ = OH, C₁-C₈ alkoxy, OCH₂O
G = Z—Ar, Y—Ar₂
Z = O(CH₂)ₘ, CONH(CH₂)ₘ, NHCO(CH₂)ₘ
Y = O(CH₂)ₘCH, CONH(CH₂)ₘCH, NHCO(CH₂)ₘCH
n = 1-5; m = 1-3; Ar = 5-15 membered unsaturated or aromatic ring

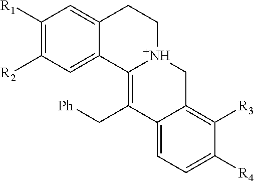

R₁, R₃, R₂, R₄ = OH, C₁-C₈ alkoxy, OCH₂O
G = Z—Ar, Y—Ar₂
Z = O(CH₂)ₘ, CONH(CH₂)ₘ, NHCO(CH₂)ₘ
Y = O(CH₂)ₘCH, CONH(CH₂)ₘCH, NHCO(CH₂)ₘCH
n = 1-5; m = 1-3; Ar = 5-15 membered unsaturated or aromatic ring

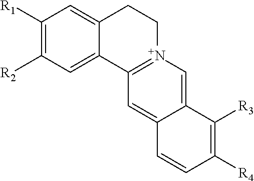

R₁, R₂, R₃, R₄ = OCH₃, OH, OCH₂O

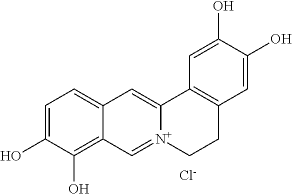 [SMILES: Oc1cc2c(cc1O)-c1cc3ccc(O)c(O)c3c[n+]1CC2 ; [Cl-]]

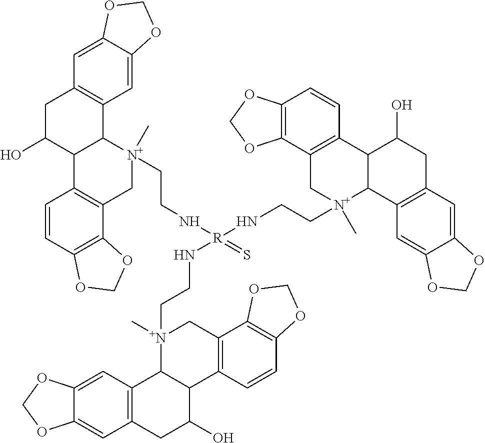

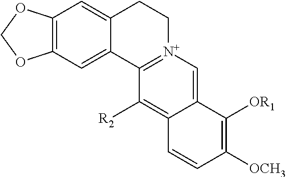

R₁ = H, Me
R₂ = Bn, 3,5-dinitrobenzyl

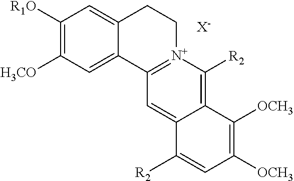

X = F, Cl, Br, I, SO₄ NO₃, PO₄, citrate, acetate, lactate
R₁ and R₂ = independently alkyl; R₃ = H, F, Cl, Br, or I

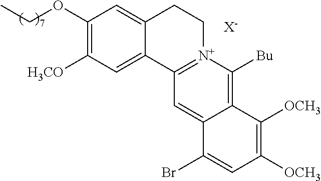

X = F, Cl, Br, I, SO₄ NO₃, PO₄, citrate, acetate, lactate
R₁ and R₂ = independently alkyl; R₃ = H, F, Cl, Br, or I

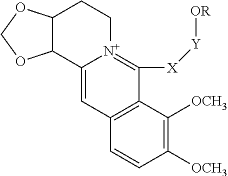

Y = CH₂, —C═O, —C═S; X = C having a linear, branched,
saturated/unsaturated linear structure; n = 1-10

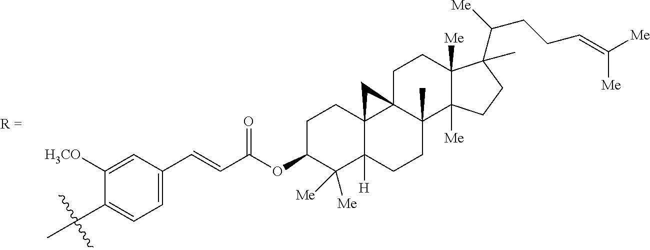

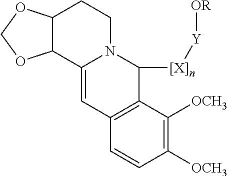

Y = CH₂, —C═O, —C═S; X = C having a linear, branched, saturated/unsaturated
linear structure; n = 1-10

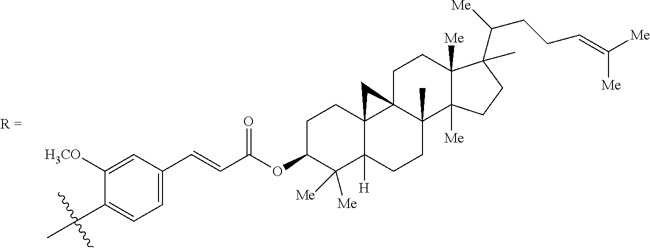

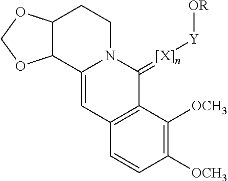

Y = CH₂, —C═O, —C═S; X = C having a linear, branched, saturated/unsaturated
linear structure; n = 1-10

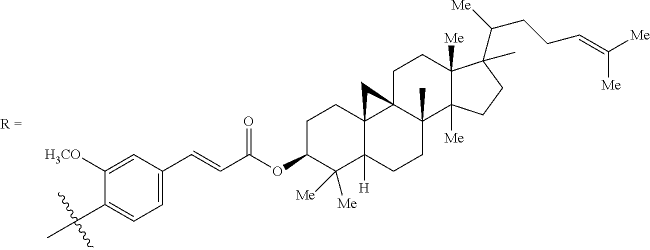

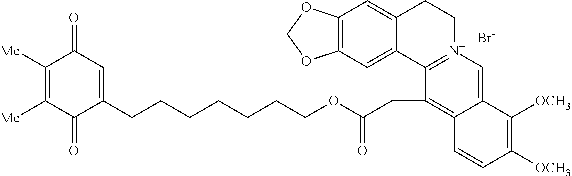 [SMILES: COc1ccc2c(CC(=O)OCCCCCCCC3=CC(=O)C(C)=C(C)C3=O)c3[n+](cc2c1OC)CCc1cc2c(cc1-3)OCO2 ; [Br-]]

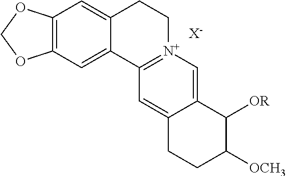

R = glucosyl, mannosyl, maltosyl, lactosyl, galactosyl, fructosyl, xylosyl, arabinosyl
X = Cl, Br, I

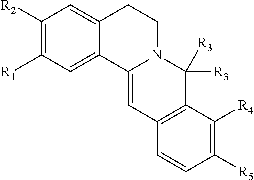

R₁, R₂ = H, C₁-C₄ alkoxy, OCH₂O
R₃ = C₁-C₃ alkyl
R₄, R₅ = C₁-C₂ alkoxy

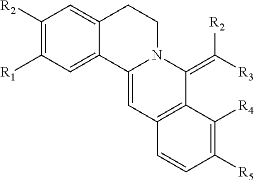

R₁, R₂ = H, C₁-C₄ alkoxy, OCH₂O
R₃ = CN, COOR₆ (R₆ = C₁-C₂ alkyl)
R₄, R₅ = C₁-C₂ alkoxy

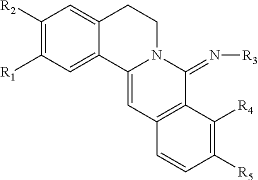

R₁, R₂ = H, C₁-C₄ alkoxy, OCH₂O
R₃ = C₁-C₂ alkyl, phenyl
R₄, R₅ = C₁-C₂ alkoxy

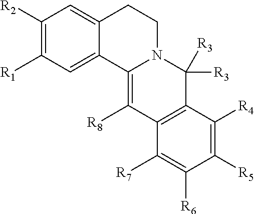

R₁, R₂ = H, (CH₂)₀₋₆, CO₂R′, C(O)R″, OR′, NR₁₀R₁₁, C(O)NR₁₀R₁₁, alkyl
R₁R₂ = OCH₂CH₂O; R₃, R₈ = H, OH, Cl, Br, F, I, CN, NH₂, (:O)NH₂, CO₂H,
alkyl; R₃′ = H; R₃R₃′ = O; R₄ = H, halogen, OR′, OSO₂R″, OC(:O)R″, OCO2R″
OC(O)NR′R″, O-alkylene-NR′R″, O-alkylene-OSO₂R″, O-alkylene-NR′SO₂R″,
O-alkylene-NR′COR′, alkyl: R₅, R₆ = H, halogen, OH, alkoxy
R₄R₅ = OCH₂O; R₅R₆ = OCH₂O: R₇ = H, OH, halogen, alkyl or alkoxy
R₁₀, R₁₁ = H, CO₂R″, alkyl

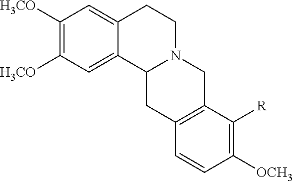

R = SO₂C₆H₄-3-F

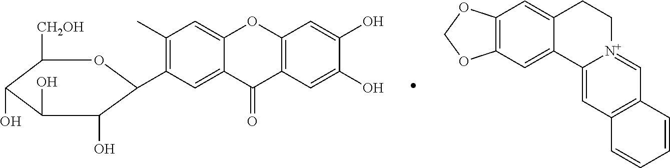

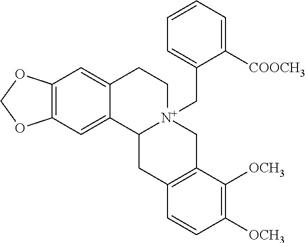 [SMILES: COC(=O)c1ccccc1C[N+]12CCc3cc4c(cc3C1Cc1ccc(OC)c(OC)c1C2)OCO4]

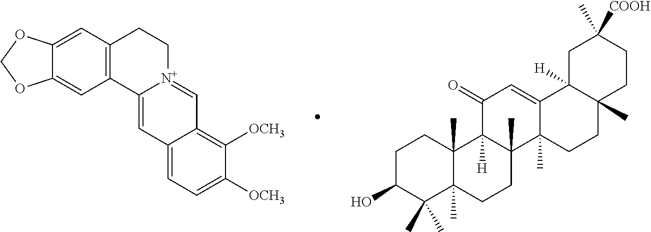

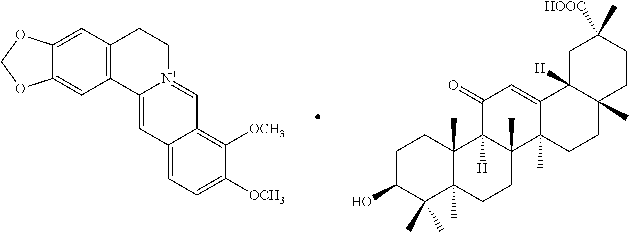

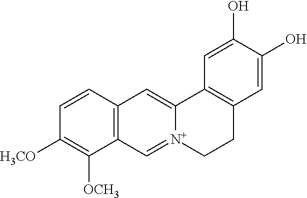 [SMILES: COc1ccc2cc3[n+](cc2c1OC)CCc1cc(O)c(O)cc1-3]

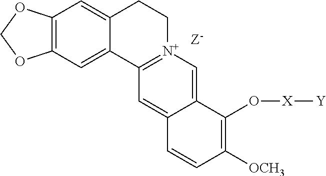

X = (CH₂)ₙ, (CH₂)ₘCO; n = 2-10; m = 1-9
Y = NR₁Ar, OAr; Ar = substituted aryl
R₁ = H, Me, Et, Pr, i-Pr; Z = F, Cl, Br, I

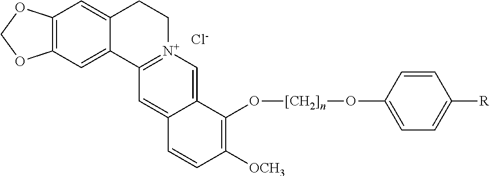

R = 2-acetic acid Me ester, 3-acetic Me ester, 4-acetic Me ester, 2-acetic Me Et
ester, 3-acetic Me Et ester, 4-acetic Me Et ester, 2-acetate, 3-acetate, 4-acetate,
2-acetate potassium, 3-acetate potassium, 4-acetate potassium; n = 2-6

In certain embodiments of the compound, X is a moiety derived from berberine or a derivative or analog thereof and Z is a bile acid or a derivative or analog thereof.

In certain embodiments of the compound, X is a moiety derived from berberine or a derivative or analog thereof and Z is a fatty acid or a derivative or analog thereof.

In certain embodiments of the compound, X is a moiety derived from berberine or a derivative or analog thereof and Z is a rhein, or R-(+)-α-lipoic, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof.

In certain embodiments of the compound, X is a moiety derived from L-carnitine or a derivative or analog thereof selected from Table 4.

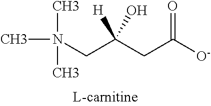

L-Carnitine is a naturally occurring amino acid. It is biosynthesized in the liver and kidneys from lysine and methionine. L-Carnitine plays an important role in the metabolism of fat, functioning as a transporter of fatty acids into the mitochondria. Exemplary L-carnitine derivatives or analogs are listed in Table 4.

TABLE 4
L-carnitine Derivatives or Analogs

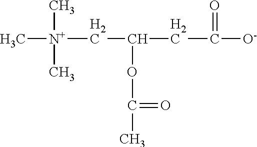

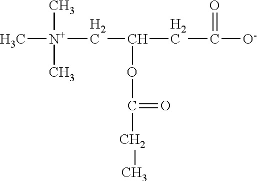

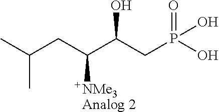

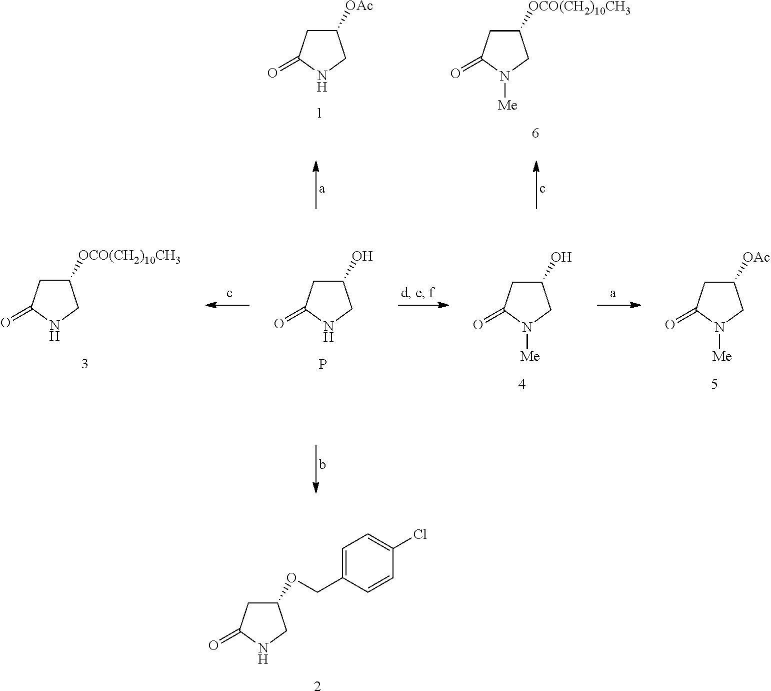

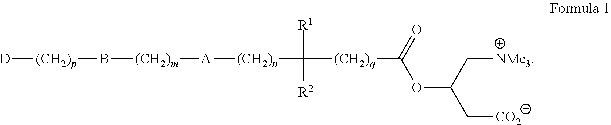

Wherein A is selected from the group consisting of a single bond, ‘Of, or iCH2i; m and n vary
independently and are an integer from 1 to 15; p and q vary independently from 0 to 1; B is iCR3R4; D is
selected from the group consisting of iCOzRs, ADR6, ADCOR7, iSO3R8, iSO2NH2,
iOPO(OR9)(OR1O), A)PO(OR9)(NH2), iOPO(OR9)i
OiPO(OR1O)(OR11), wherein R1 to R4 are independently selected from C1-C6 alkyl; and R5 to R11 are
independently selected from the group consisting
of hydrogen; C1-C6 alkyl; C3-C6 cycloalkyl; C2-C6 alkenyl; C6 alkynyl; C5-C10 aryl unsubstituted or
substituted With C1-C6 alkyl, hydroxyl, C1-C6 alkoxyl, 1,3-dioxolanyl, cyano, halo, nitro, trihaloalkyl,
carboxyl, C1-C6 acyl, C1-C6 hydroxyalkyl, amino, C1-C6 alkylamino, C1-C6 dialkylamino, C1-C6
acylamino, C1-C6 alkoxylcarbonyl; C5-C6 arylalkyl unsubstituted or substituted With C1-C6 alkyl,
hydroxyl, C1-C6 alkoxyl, 1,3-dioxolanyl, cyano, halo, trihaloalkyl, carboxyl, C1-C6 acyl, C1-C6
hydroxyalkyl, amino, C1-C6 alkylamino, C1-C6 dialkylamino, C1-C6 alkoxylcarbonyl; C1-C6
carboxyalkyl; C1-C6 acylamino; C1-C6 sulfonatoalkyl; C1-C6 sulfamylalkyl; and C1-C6
phosphonatoalkyl.

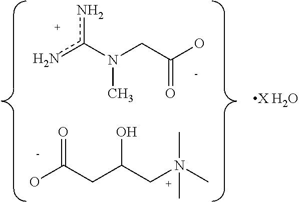

Wherein X is an integer betWeen about 0 and 5

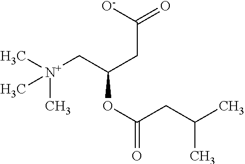 [SMILES: CC(C)CC(=O)O[C@H](CC(=O)[O-])C[N+](C)(C)C]

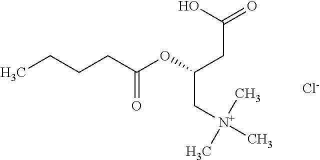 [SMILES: CCCCC(=O)O[C@H](CC(=O)O)C[N+](C)(C)C ; [Cl-]]

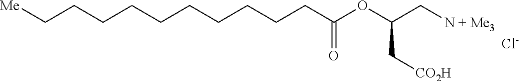

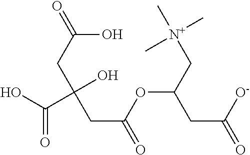 [SMILES: C[N+](C)(C)CC(CC(=O)[O-])OC(=O)CC(O)(CC(=O)O)C(=O)O]

In certain embodiments of the compound, X is a moiety derived from L-carnitine or a derivative or analog thereof and Z is a bile acid or a derivative or analog thereof.

In certain embodiments of the compound, X is a moiety derived from L-carnitine or a derivative or analog thereof and Z is a fatty acid or a derivative or analog thereof.

In certain embodiments of the compound, X is a moiety derived from L-carnitine or a derivative or analog thereof and Z is a rhein, or R-(+)-α-lipoic, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof.

In certain embodiments of the compound, X is a moiety derived from metformin or a derivative or analog thereof selected from Table 5.

Metformin (N,N-Dimethylimidodicarbonimidic diamide) is a potent anti-hyperglycemic agent now recommended as the first line oral therapy for type 2 diabetes (T2D). The main effect of this drug is to acutely decrease hepatic glucose production, mostly through a mild and transient inhibition of the mitochondrial respiratory-chain complex 1. In addition, the resulting decrease in hepatic energy status activates the AMP-activated protein kinase (AMPK), a cellular metabolic sensor, providing a generally accepted mechanism for metformin action on hepatic gluconeogenic program. Beyond its effect on glucose metabolism, metformin was reported to restore ovarian function in polycystic ovary syndrome, reduce fatty liver and to lower microvascular and macrovascular complications associated with T2D. Its use was also recently suggested as an adjuvant treatment for cancer or gestational diabetes, and for the prevention in pre-diabetic populations. Studies of metformin for NAFLD and NASH have multiplied in the past few years, however, its efficacy for NAFLD and NASH remains to be approved.

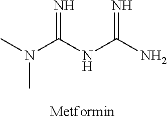

TABLE 5

| Metformin Derivatives or Analogs | |
|---|---|
| 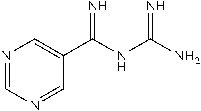 [SMILES: N=C(N)NC(=N)c1cncnc1] | |
| 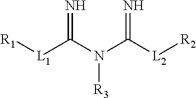 | (I) |
| L¹ and L² are independently a bond or —NH—C(NH)—; | |
| R¹ is —NR^{1A}R^{1B}, substituted or unsubstituted alkyl, substituted or | |
| unsubstituted heteroalkyl, substituted or unsubstituted cycloalkyl, | |
| substituted or unsubstituted heterocycloalkyl, substituted or | |
| unsubstituted aryl, or substituted or unsubstituted heteroaryl, | |
| wherein R^{1A} and R^{1B} are optionally joined together to form | |
| a substituted or unsubstituted hcterocycloalkyl; R² is —NR^{2A}R^{2B}, | |
| substituted or unsubstituted alkyl, substituted or unsubstituted | |
| heteroalkyl, substituted or unsubstituted cycloalkyl, substituted | |
| or unsubstituted heterocycloalkyl, substituted or unsubstituted aryl, | |
| or substituted or unsubstituted heteroaryl, wherein R^{2A} and | |
| R^{2B} are optionally joined together to form a substituted or | |
| unsubstituted heterocycloalkyl; R^{1A}, R^{1B}, R^{2A}, and R^{2B} are | |
| independently hydrogen. —OR⁴, substituted or unsubstituted alkyl, | |
| substituted or unsubstituted heteroalkyl, substituted or | |
| unsubstituted cycloalkyl, substituted or unsubstituted | |
| heterocycloalkyl, substituted or unsubstituted aryl, or | |
| substituted or unsubstituted heteroaryl; R³ is hydrogen or | |
| unsubstituted C1-C5 alkyl; and R⁴ is hydrogen, substituted or | |
| unsubstituted alkyl, substituted or unsubstituted heteroalkyl, | |
| substituted or unsubstituted cycloalkyl, substituted or | |
| unsubstituted heterocycloalkyl, substituted or unsubstituted aryl, | |
| or substituted or unsubstituted heteroaryl; | |
| 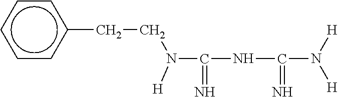 | |
| 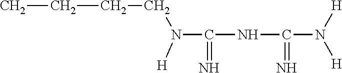 | |
| 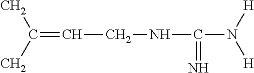 | |
| 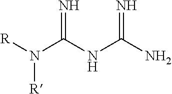 | |
| R′ = —H, —Ph, substituted —Ph | |
| R = R₁ substituted —Ph | |
| R₁ = C₁-C₆ alkyl, C₁-C₄ alkyl, fluorinated alkyl, acyl, ester, aryl, | |
| halogen, NO₂, NH₂, —H, —OR₂, —SR₂ | |
| R₂ = C₁-C₆ alkyl, C₁-C₄ fluorinated alkyl, acyl | |

In certain embodiments of the compound, X is a moiety derived from metformin or a derivative or analog thereof and Z is a bile acid or a derivative or analog thereof.

In certain embodiments of the compound, X is a moiety derived from metformin or a derivative or analog thereof and Z is a fatty acid or a derivative or analog thereof.

In certain embodiments of the compound, X is a moiety derived from metformin or a derivative or analog thereof and Z is a rhein, or R-(+)-α-lipoic, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof.

In certain preferred embodiments of the compound, Z is a moiety derived from ursodeoxycholic acid and X is a moiety derived from berberine.
BBR—Y-UDCA

In certain preferred embodiments of the compound, Z is a moiety derived from ursodeoxycholic acid and X is a moiety derived from L-carnitine.
(L-carnitine)-Y-UDCA

In certain preferred embodiments of the compound, Z is a moiety derived from ursodeoxycholic acid and X is a moiety derived from one of metformin, coptisine, palmatine and jatrorrhizine.
Metformin-Y-UDCA
Coptisine-Y-UDCA
Palmatine-Y-UDCA
Jatrorrhizine-Y-UDCA

Beyond its effect on glucose metabolism, metformin was reported to restore ovarian function in polycystic ovary syndrome, reduce fatty liver and to lower microvascular and macrovascular complications associated with T2D. Its use was also recently suggested as an adjuvant treatment for cancer or gestational diabetes, and for the prevention in pre-diabetic populations. Studies of metformin for NAFLD and NASH have multiplied in the past few years, however, its efficacy for NAFLD and NASH remains to be approved.

Coptisine [6,7-Dihydro-bis(1,3)benzodioxolo (5,6-a:4′,5′-g)quinolizinium], palmatine [2,3,9,10-tetramethoxy-5,6-dihydroisoquinolino[2,1-b]isoquinolin-7-ium], and jatrorrhizine [2,9,10-trimethoxy-5,6-dihydroisoquinolino[2,1-b]isoquinolin-7-ium-3-ol] are naturally alkaloids that have demonstrated similar pharmacological properties as berberine in previous studies.

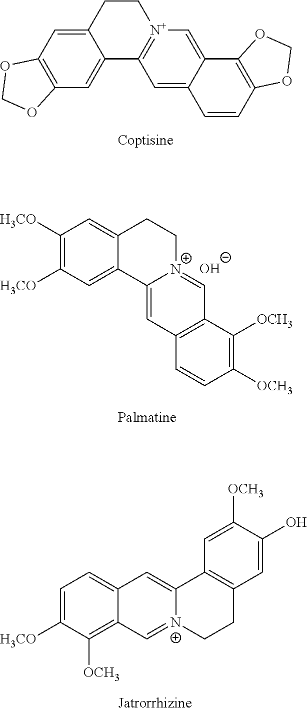

In certain embodiments of the compound, Z is a moiety derived from ursodeoxycholic acid, or a derivative or analog thereof, and X is a moiety derived from an unsaturated fatty acid. In certain embodiments of the compound, the unsaturated fatty acid is selected from Table 6.

TABLE 6
Unsaturated Fatty Acids

| Common Name | Chemical Structure |
|---|---|
| Myristoleic acid | CH₃(CH₂)₃CH═CH(CH₂)₇COOH |
| Palmitoleic acid | CH₃(CH₂)₅CH═CH(CH₂)₇COOH |
| Sapienic acid | CH₃(CH₂)₈CH═CH(CH₂)₄COOH |
| Oleic acid | CH₃(CH₂)₇CH═CH(CH₂)₇COOH |
| Elaidic acid | CH₃(CH₂)₇CH═CH(CH₂)₇COOH |
| Vaccenic acid | CH₃(CH₂)₅CH═CH(CH₂)₉COOH |
| Linoleic acid | CH₃(CH₂)₄CH═CHCH₂CH═CH(CH₂)₇COOH |
| Linoelaidic acid | CH₃(CH₂)₄CH═CHCH₂CH═CH(CH₂)₇COOH |
| α-Linolenic acid | CH₃CH₂CH═CHCH₂CH═CHCH₂CH═CH(CH₂)₇COOH |
| Arachidonic acid | CH₃(CH₂)₄CH═CHCH₂CH═CHCH₂CH═CHCH₂CH═CH(CH₂)₃COOH |
| Eicosapentaenoic acid | CH₃CH₂CH═CHCH₂CH═CHCH₂CH═CHCH₂CH═CHCH₂CH═CH(CH₂)₃COOH |
| Erucic acid | CH₃(CH₂)₇CH═CH(CH₂)₁₁COOH |
| Docosahexaenoic acid | CH₃CH₂CH═CHCH₂CH═CHCH₂CH═CHCH₂CH═CHCH₂CH═CHCH₂CH═CH(CH₂)₂COOH |

In certain embodiments of the compound, Z is a moiety derived from ursodeoxycholic acid, or a derivative or analog thereof, and X is a moiety derived from eicosapentaenoic acid (EPA).
EPA-Y-UDCA

In certain embodiments of the compound, Z is a moiety derived from ursodeoxycholic acid, or a derivative or analog thereof, and X is a moiety derived from docosahexaenoic (DHA).
DHA-Y-UDCA

In certain embodiments of the compound, the linker includes an amide bond or an ester bond. In certain preferred embodiments, the linker includes an amide bond. In certain embodiments, the linker includes a moiety derived from a natural or synthetic amino acid, for example, selected from Table 7. In certain preferred embodiments, the linker includes an ester bond.

TABLE 7
Common Amino Acids

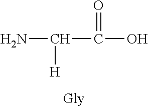

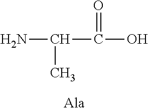

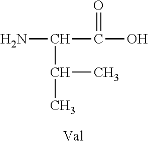

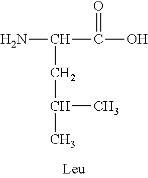

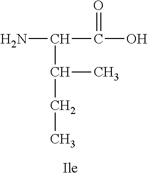

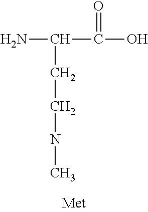

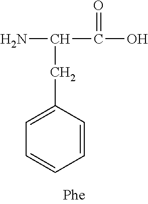 [SMILES: NC(Cc1ccccc1)C(=O)O]

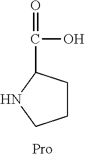

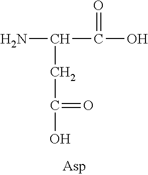

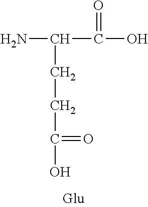

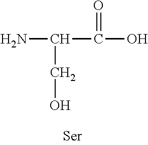

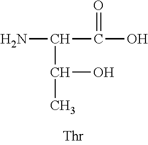

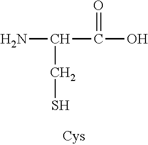

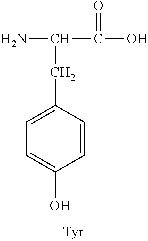 [SMILES: NC(Cc1ccc(O)cc1)C(=O)O]

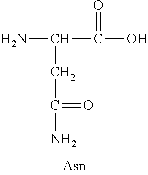

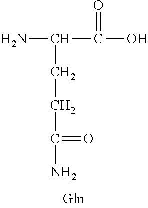

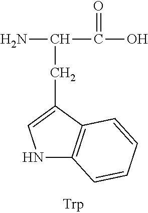 [SMILES: NC(Cc1c[nH]c2ccccc12)C(=O)O]

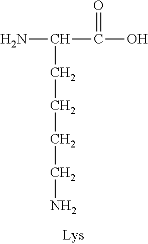

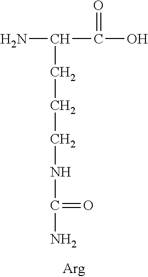 [SMILES: NC(=O)NCCCC(N)C(=O)O]

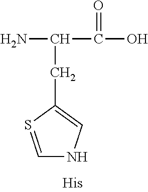

In another aspect, the invention generally relates to a pharmaceutical composition comprising an amount of a compound having the formula of:

X—Y—Z (I)

wherein

(a) X is a moiety derived from a pharmacologically active organic base or acid;

(b) Z is a moiety derived from a pharmacologically active organic acid; and

(c) Y a covalent bond or a linker,

wherein at least

X is a moiety derived from berberine or a derivative or analog thereof, or L-carnitine or a derivative or analog thereof, or metformin or a derivative or analog thereof, or unsaturated fatty acid or a derivative or analog thereof,

Z is moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, wherein the amount is effective to treat, prevent, or reduce one or more diseases or disorders selected from liver diseases or disorders, diabetes, diabetic complications, pre-diabetes, dyslipidemia, obesity, metabolic syndromes, muscle atrophy, inflammation, and cancers or a related disease or disorder thereof in a mammal including a human, and a pharmaceutically acceptable excipient, carrier, or diluent.

In certain embodiments of the pharmaceutical composition, the disease or disorder is selected from NAFLD, NASH, cholestatic liver diseases or graft-versus-host disease of the liver.

In certain embodiments of the pharmaceutical composition, the disease or disorder is selected from diabetes, diabetic complications and pre-diabetes.

In certain embodiments of the pharmaceutical composition, the disease or disorder is dyslipidemia.

In certain embodiments of the pharmaceutical composition, the disease or disorder is obesity.

In certain embodiments of the pharmaceutical composition, the disease or disorder is metabolic syndromes.

In certain embodiments of the pharmaceutical composition, the disease or disorder is muscle atrophy.

In certain embodiments of the pharmaceutical composition, the disease or disorder is inflammation.

In certain embodiments of the pharmaceutical composition, the disease or disorder is cancer.

In certain embodiments of the pharmaceutical composition, Z is a moiety derived from ursodeoxycholic acid, or a derivative or analog thereof selected from Table 1.

In certain embodiments of the pharmaceutical composition, Z is a moiety derived from eicosapentaenoic acid or docosahexaenoic acid, or a derivative or analog thereof.

In certain embodiments of the pharmaceutical composition, Z is a moiety derived from rhein or a derivative or analog thereof.

In certain embodiments of the pharmaceutical composition, Z is a moiety derived from R-(+)-α-lipoic acid or a derivative or analog thereof.

In certain embodiments of the pharmaceutical composition, Z is a moiety derived from ursolic acid or corosolic acid or a derivative or analog thereof.

In certain embodiments of the pharmaceutical composition, Z is a moiety derived from hydroxycitric acid or a derivative or analog thereof.

In certain embodiments of the pharmaceutical composition, Z is a moiety selected from a pharmaceutically active organic acid from Table 2.

In certain embodiments of the pharmaceutical composition, X is a moiety derived from berberine or a derivative or analog thereof selected from Table 3

In certain embodiments of the compound, X is a moiety derived from L-carnitine or a derivative or analog thereof selected from Table 4.

In certain embodiments of the compound, X is a moiety derived from metformin or a derivative or analog thereof selected from Table 5.

In certain embodiments of the pharmaceutical composition, X is a moiety derived from berberine, or a derivative or analog thereof, and Z is a bile acid, or a derivative or analog thereof.

In certain embodiments of the pharmaceutical composition, X is a moiety derived from berberine, or a derivative or analog thereof, and Z is a fatty acid, or a derivative or analog thereof.

In certain embodiments of the pharmaceutical composition, X is a moiety derived from L-carnitine, or a derivative or analog thereof, and Z is a bile acid, or a derivative or analog thereof.

In certain embodiments of the pharmaceutical composition, X is a moiety derived from L-carnitine, or a derivative or analog thereof, and Z is a fatty acid, or a derivative or analog thereof.

In certain embodiments of the pharmaceutical composition, X is a moiety derived from metformin, or a derivative or analog thereof, and Z is a bile acid, or a derivative or analog thereof.

In certain embodiments of the pharmaceutical composition, X is a moiety derived from metformin, or a derivative or analog thereof, and Z is a fatty acid, or a derivative or analog thereof.

In certain embodiments of the pharmaceutical composition, Z is a moiety derived from ursodeoxycholic acid and X is a moiety derived from berberine. In certain embodiments of the compound, Z is a moiety derived from ursodeoxycholic acid and X is a moiety derived from L-carnitine. In certain embodiments of the compound, Z is a moiety derived from ursodeoxycholic acid and X is a moiety derived from one of metformin, coptisine, palmatine and jatrorrhizine. In certain embodiments of the compound, Z is moiety derived from ursodeoxycholic acid or a derivative or analog and X is a moiety derived from an unsaturated fatty acid. In certain embodiments of the compound, the unsaturated fatty acid is selected from Table 6.

In certain embodiments of the pharmaceutical composition, the linker includes an amide bond or an ester bond. In certain preferred embodiments, the linker includes an amide bond. In certain embodiments, the linker includes a moiety derived from an amino acid selected from Table 7. In certain preferred embodiments, the linker comprises an ester bond.

The pharmaceutical composition may further include one or more of vitamin E, omega-3 fatty acids, S-adenosylmethionine, N-acetyl cysteine, silymarin, polyenylphosphatidylcholine, resveratrol, and vitamin D.

In yet another aspect, the invention generally relates to a method for treating, reducing, or preventing a disease or disorder. The method includes: administering to a subject in need thereof a pharmaceutical composition comprising an amount of a compound having the formula of:

X—Y—Z (I)

wherein

(a) X is a moiety derived from a pharmacologically active organic base or acid;

(b) Z is a moiety derived from a pharmacologically active organic acid; and

(c) Y a covalent bond or a linker,

wherein at least

Z is moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof,

X is a moiety derived from berberine or a derivative or analog thereof, or L-carnitine or a derivative or analog thereof, or metformin or a derivative or analog thereof, wherein the amount is effective to treat, prevent, or reduce one or more diseases or disorders selected from liver diseases or disorders, diabetes, diabetic complications, pre-diabetes, dyslipidemia, obesity, metabolic syndromes, muscle atrophy, inflammation, and cancers, or a related disease or disorder thereof in a mammal, including a human, and a pharmaceutically acceptable excipient, carrier, or diluent.

In certain embodiments of the method, the disease or disorder is selected from NAFLD, NASH, cholestatic liver diseases or graft-versus-host disease of the liver.

In certain embodiments of the method, the disease or disorder is selected from diabetes, diabetic complications and pre-diabetes.

In certain embodiments of the method, the disease or disorder is dyslipidemia.

In certain embodiments of the method, the disease or disorder is obesity.

In certain embodiments of the method, the disease or disorder is metabolic syndromes.

In certain embodiments of the method, the disease or disorder is muscle atrophy.

In certain embodiments of the method, the disease or disorder is inflammation.

In certain embodiments of the method, the disease or disorder is cancer.

In certain embodiments of the method, Z is a moiety derived from ursodeoxycholic acid, or a derivative or analog thereof selected from Table 1.

In certain embodiments of the pharmaceutical composition, Z is a moiety derived from eicosapentaenoic acid or docosahexaenoic acid, or a derivative or analog thereof.

In certain embodiments of the pharmaceutical composition, Z is a moiety derived from rhein or a derivative or analog thereof.

In certain embodiments of the pharmaceutical composition, Z is a moiety derived from R-(+)-α-lipoic acid or a derivative or analog thereof.

In certain embodiments of the pharmaceutical composition, Z is a moiety derived from ursolic acid or corosolic acid or a derivative or analog thereof.

In certain embodiments of the pharmaceutical composition, Z is a moiety derived from hydroxycitric acid or a derivative or analog thereof.

In certain embodiments of the pharmaceutical composition, Z is a moiety selected from a pharmacologically active organic acid from Table 2.

In certain embodiments of the method, X is a moiety derived from berberine, or a derivative or analog thereof selected from Table 3. In certain embodiments of the compound, X is a moiety derived from L-carnitine, or a derivative or analog thereof selected from Table 4. In certain embodiments of the compound, X is a moiety derived from metformin, or a derivative or analog thereof selected from Table 5.

In certain embodiments of the method, X is a moiety derived from berberine, or a derivative or analog thereof, and Z is a bile acid, or a derivative or analog thereof.

In certain embodiments of the method, X is a moiety derived from berberine, or a derivative or analog thereof, and Z is a fatty acid, or a derivative or analog thereof.

In certain embodiments of the pharmaceutical composition, X is a moiety derived from L-carnitine, or a derivative or analog thereof, and Z is a bile acid, or a derivative or analog thereof.

In certain embodiments of the method, X is a moiety derived from L-carnitine, or a derivative or analog thereof, and Z is a fatty acid, or a derivative or analog thereof.

In certain embodiments of the method, X is a moiety derived from metformin, or a derivative or analog thereof, and Z is a bile acid, or a derivative or analog thereof.

In certain embodiments of the method, X is a moiety derived from metformin, or a derivative or analog thereof, and Z is a fatty acid, or a derivative or analog thereof.

In certain embodiments of the method, Z is a moiety derived from ursodeoxycholic acid and X is a moiety derived from berberine. In certain embodiments of the compound, Z is a moiety derived from ursodeoxycholic acid and X is a moiety derived from L-carnitine. In certain embodiments of the compound, Z is a moiety derived from ursodeoxycholic acid and X is a moiety derived from one of metformin, coptisine, palmatine and jatrorrhizine.

In certain embodiments of the compound, Z is a moiety derived from ursodeoxycholic acid, or a derivative or analog thereof, and X is a moiety derived from an unsaturated fatty acid. In certain embodiments of the compound, the unsaturated fatty acid is selected from Table 6.

In certain embodiments of the method, the linker includes an amide bond or an ester bond. In certain preferred embodiments, the linker includes an amide bond. In certain embodiments, the linker includes a moiety derived from an amino acid selected from Table 6. In certain preferred embodiments, the linker includes an ester bond.

In certain embodiments of the method, the pharmaceutical composition further includes one or more of vitamin E, omega-3 fatty acids, S-adenosylmethionine, N-acetyl cysteine, silymarin, polyenylphosphatidylcholine, resveratrol, and vitamin D.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance that can be adapted to the practice of this invention in its various embodiments and equivalents thereof.

What is claimed is:

1. A compound having the formula of:
X—Y—Z (I)
wherein
(a) X is a moiety derived from a pharmacologically active organic base or acid;
(b) Z is a moiety derived from a pharmacologically active organic acid; and
(c) Y a covalent bond or a linker,
wherein at least
X is a moiety derived from berberine, or a derivative or analog thereof, or L-carnitine, or a derivative or analog thereof; or metformin, or a derivative or analog thereof; or unsaturated fatty acid, or a derivative or analog thereof;
Z is moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof.

2. The compound of claim 1, wherein the linker comprises an amide bond or an ester bond.

3. The compound of claim 1, wherein Z is moiety derived from ursodeoxycholic acid, or a derivative or analog thereof selected from Table 1.

4. The compound of claim 1, wherein X is a moiety derived from berberine, or a derivative or analog thereof selected from Table 3.

5. The compound of claim 1, wherein X is a moiety derived from L-carnitine, or a derivative or analog thereof selected from Table 4.

6. The compound of claim 1, wherein X is a moiety derived from metformin, or a derivative or analog thereof selected from Table 5.

7. The compound of claim 1, wherein X is a moiety derived from unsaturated fatty acid, or a derivative or analog thereof selected from Table 6.

8. The compound of claim 1, wherein X is a moiety derived from berberine, or a derivative or analog thereof, and Z is a bile acid, or a derivative or analog thereof.

9. The compound of claim 1, wherein X is a moiety derived from berberine, or a derivative or analog thereof, and Z is a fatty acid, or a derivative or analog thereof.

10. The compound of claim 1, wherein X is a moiety derived from berberine, or a derivative or analog thereof, and Z is rhein or a derivative or analog thereof.

11. The compound of claim 1, wherein X is a moiety derived from berberine, or a derivative or analog thereof, and Z is R-(+)-α-lipoic acid, or a derivative or analog thereof.

12. The compound of claim 1, wherein X is a moiety derived from berberine, or a derivative or analog thereof, and Z is ursolic acid or corosolic acid or a derivative or analog thereof.

13. The compound of claim 1, wherein X is a moiety derived from berberine, or a derivative or analog thereof, and Z is hydroxycitric acid or a derivative or analog thereof.

14. The compound of claim 1, wherein X is a moiety derived from berberine, or a derivative or analog thereof, and Z is a moiety selected from a pharmacologically active organic acid from Table 2.

15. The compound of claim 1, wherein X is a moiety derived from L-carnitine, or a derivative or analog thereof, and Z is a bile acid, or a derivative or analog thereof.

16. The compound of claim 1, wherein X is a moiety derived from L-carnitine, or a derivative or analog thereof, and Z is a fatty acid, or a derivative or analog thereof.

17. The compound of claim 1, wherein X is a moiety derived from L-carnitine, or a derivative or analog thereof, and Z is rhein or a derivative or analog thereof.

18. The compound of claim 1, wherein X is a moiety derived from L-carnitine, or a derivative or analog thereof, and Z is R-(+)-α-lipoic acid, or a derivative or analog thereof.

19. The compound of claim 1, wherein X is a moiety derived from L-carnitine, or a derivative or analog thereof, and Z is ursolic acid or corosolic acid or a derivative or analog thereof.

20. The compound of claim 1, wherein X is a moiety derived from L-carnitine, or a derivative or analog thereof, and Z is hydroxycitric acid or a derivative or analog thereof.

21. The compound of claim 1, wherein X is a moiety derived from L-carnitine, or a derivative or analog thereof, and Z is a moiety selected from a pharmacologically active organic acid from Table 2.

22. The compound of claim 1, wherein Z is a moiety derived from ursodeoxycholic acid and X is a moiety derived from berberine.

23. The compound of claim 1, wherein Z is a moiety derived from ursodeoxycholic acid and X is a moiety derived from L-carnitine.

24. The compound of claim 1, wherein Z is a moiety derived from ursodeoxycholic acid and X is a moiety derived from one of metformin, coptisine, palmatine and jatrorrhizine.

25. The compound of claim 1, wherein Z is a moiety derived from ursodeoxycholic acid, or a derivative or analog thereof, and X is a moiety derived from an unsaturated fatty acid.

26. The compound of claim 25, wherein the unsaturated fatty acid is selected from Table 6.

27. The compound of any of claims 3-25, wherein the linker comprises an amide bond.

28. The compound of claim 27, wherein the linker comprises a moiety derived from an amino acid selected from Table 6.

29. The compound of any of claims 3-25, wherein the linker comprises an ester bond.

30. A pharmaceutical composition comprising an amount of a compound having the formula of:
X—Y—Z (I)
wherein
(a) X is a moiety derived from a pharmacologically active organic base or acid;
(b) Z is a moiety derived from a pharmacologically active organic acid; and
(c) Y a covalent bond or a linker,
wherein at least
X is a moiety derived from berberine, or a derivative or analog thereof, or L-carnitine, or a derivative or analog thereof; or metformin, or a derivative or analog thereof; or unsaturated fatty acid, or a derivative or analog thereof
Z is moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, effective to treat, prevent, or reduce one or more diseases or disorders selected from liver diseases or disorders, diabetes, diabetic complications, pre-diabetes, dyslipidemia, obesity, metabolic syndromes, muscle atrophy, inflammation, and cancers or a related disease or disorder thereof in a mammal including a human, and a pharmaceutically acceptable excipient, carrier, or diluent.

31. The pharmaceutical composition of claim 30, wherein the disease or disorder is selected from non-alcoholic fatty liver disease (NAFLD), non-alcoholic steatohepatitis (NASH), cholestatic liver diseases or graft-versus-host disease of the liver.

32. The pharmaceutical composition of claim 30, wherein the disease or disorder is selected from diabetes, diabetic complications and pre-diabetes.

33. The pharmaceutical composition of claim 30, wherein the disease or disorder is dyslipidemia.

34. The pharmaceutical composition of claim 30, wherein the disease or disorder is obesity.

35. The pharmaceutical composition of claim 30, wherein the disease or disorder is metabolic syndromes.

36. The pharmaceutical composition of claim 30, wherein the disease or disorder is muscle atrophy.

37. The pharmaceutical composition of claim 30, wherein the disease or disorder is inflammation.

38. The pharmaceutical composition of claim 30, wherein the disease or disorder is cancer.

39. The pharmaceutical composition of claim 30, wherein the linker comprises an amide bond or an ester bond.

40. The pharmaceutical composition of claim 30, wherein Z is a moiety derived from ursodeoxycholic acid, or a derivative or analog thereof selected from Table 1.

41. The pharmaceutical composition of claim 30, wherein X is a moiety derived from berberine, or a derivative or analog thereof selected from Table 3.

42. The pharmaceutical composition of claim 30, wherein X is a moiety derived from L-carnitine, or a derivative or analog thereof selected from Table 4.

43. The pharmaceutical composition of claim 30, wherein X is a moiety derived from metformin, or a derivative or analog thereof selected from Table 5.

44. The pharmaceutical composition of claim 30, wherein X is a moiety derived from berberine, or a derivative or analog thereof, and Z is a bile acid, or a derivative or analog thereof.

45. The pharmaceutical composition of claim 30, wherein X is a moiety derived from berberine, or a derivative or analog thereof, and Z is a fatty acid, or a derivative or analog thereof.

46. The pharmaceutical composition of claim 30, wherein X is a moiety derived from berberine, or a derivative or analog thereof, and Z is rhein or a derivative or analog thereof.

47. The pharmaceutical composition of claim 30, wherein X is a moiety derived from berberine, or a derivative or analog thereof, and Z is R-(+)-α-lipoic acid, or a derivative or analog thereof.

48. The pharmaceutical composition of claim 30, wherein X is a moiety derived from berberine, or a derivative or analog thereof, and Z is ursolic acid or corosolic acid or a derivative or analog thereof.

49. The pharmaceutical composition of claim 30, wherein X is a moiety derived from berberine, or a derivative or analog thereof, and Z is hydroxycitric acid or a derivative or analog thereof.

50. The pharmaceutical composition of claim 30, wherein X is a moiety derived from berberine, or a derivative or analog thereof, and Z is a moiety selected from a pharmacologically active organic acid from Table 2.

51. The pharmaceutical composition of claim 30, wherein X is a moiety derived from L-carnitine, or a derivative or analog thereof, and Z is a bile acid, or a derivative or analog thereof.

52. The pharmaceutical composition of claim 30, wherein X is a moiety derived from L-carnitine, or a derivative or analog thereof, and Z is a fatty acid, or a derivative or analog thereof.

53. The pharmaceutical composition of claim 30, wherein X is a moiety derived from L-carnitine, or a derivative or analog thereof, and Z is rhein or a derivative or analog thereof.

54. The pharmaceutical composition of claim 30, wherein X is a moiety derived from L-carnitine, or a derivative or analog thereof, and Z is R-(+)-α-lipoic acid, or a derivative or analog thereof.

55. The pharmaceutical composition of claim 30, wherein X is a moiety derived from L-carnitine, or a derivative or analog thereof, and Z is ursolic acid or corosolic acid or a derivative or analog thereof.

56. The pharmaceutical composition of claim 30, wherein X is a moiety derived from L-carnitine, or a derivative or analog thereof, and Z is hydroxycitric acid or a derivative or analog thereof.

57. The pharmaceutical composition of claim 30, wherein X is a moiety derived from L-carnitine, or a derivative or analog thereof, and Z is a moiety selected from a pharmacologically active organic acid from Table 2.

58. The pharmaceutical composition of claim 30, wherein Z is a moiety derived from ursodeoxycholic acid and X is a moiety derived from berberine.

59. The pharmaceutical composition of claim 30, wherein Z is a moiety derived from ursodeoxycholic acid and X is a moiety derived from L-carnitine.

60. The pharmaceutical composition of claim 30, wherein Z is a moiety derived from ursodeoxycholic acid and X is a moiety derived from one of metformin, coptisine, palmatine and jatrorrhizine.

61. The pharmaceutical composition of claim 30, wherein Z is moiety derived from ursodeoxycholic acid, or a derivative or analog, and X is a moiety derived from an unsaturated fatty acid.

62. The pharmaceutical composition of claim 61, wherein the unsaturated fatty acid is selected from Table 5.

63. The pharmaceutical composition of any of claims 40-62, wherein the linker comprises an amide bond.

64. The pharmaceutical composition of claim 63, wherein the linker comprises a moiety derived from an amino acid selected from Table 6.

65. The pharmaceutical composition of any of claims 40-62, wherein the linker comprises an ester bond.

66. The pharmaceutical composition of any of claims 30-65, further comprising one or more of vitamin E, omega-3 fatty acids, S-adenosylmethionine, N-acetyl cysteine, silymarin, polyenylphosphatidylcholine, resveratrol, and vitamin D.

67. A method for treating, reducing, or preventing a disease or disorder, comprising
administering to a subject in need thereof a pharmaceutical composition comprising an amount of a compound having the formula of:
X—Y—Z (I)
wherein
(a) X is a moiety derived from a pharmacologically active organic base or acid;
(b) Z is a moiety derived from a pharmacologically active organic acid; and
(c) Y a covalent bond or a linker,
wherein at least
X is a moiety derived from berberine, or a derivative or analog thereof, or L-carnitine, or a derivative or analog thereof; or metformin, or a derivative or analog thereof, or unsaturated fatty acid, or a derivative or analog thereof,
Z is moiety derived from ursodeoxycholic acid, or eicosapentaenoic acid, or docosahexaenoic acid, or rhein, or R-(+)-α-lipoic acid, or ursolic acid, or corosolic acid, or hydroxycitric acid, or cinnamic acid, or cholic acid, or oleanolic acid, or salicylic acid, or betulinic acid, or chlorogenic acid, or caffeic acid, or bassic acid, or acetyl L-carnitine, or S-allyl cysteine sulphoxide, or S-methyl cysteine sulfoxide, or pantothenic acid, or ascorbic acid, or retinoic acid, or nicotinic acid, or biotin, or a derivative or analog thereof, effective to treat, prevent, or reduce one or more diseases or disorders selected from liver diseases or disorders, diabetes, diabetic complications, pre-diabetes, dyslipidemia, obesity, metabolic syndromes, muscle atrophy, inflammation, and cancers, or a related disease or disorder thereof in a mammal, including a human, and a pharmaceutically acceptable excipient, carrier, or diluent.

68. The method of claim 67, wherein the disease or disorder is selected from non-alcoholic fatty liver disease (NAFLD), non-alcoholic steatohepatitis (NASH), cholestatic liver diseases or graft-versus-host disease of the liver.

69. The method of claim 67, wherein the disease or disorder is selected from diabetes, diabetic complications and pre-diabetes.

70. The method of claim 67, wherein the disease or disorder is dyslipidemia.

71. The method of claim 67, wherein the disease or disorder is obesity.

72. The method of claim 67, wherein the disease or disorder is metabolic syndromes.

73. The method of claim 67, wherein the disease or disorder is muscle atrophy.

74. The method of claim 67, wherein the disease or disorder is inflammation.

75. The method of claim 67, wherein the disease or disorder is cancer.

76. The method of claim 67, wherein the linker comprises an amide bond or an ester bond.

77. The method of claim 67, wherein Z is moiety derived from ursodeoxycholic acid, or a derivative or analog thereof selected from Table 1.

78. The method of claim 67, wherein X is a moiety derived from berberine, or a derivative or analog thereof selected from Table 3.

79. The method of claim 67, wherein X is a moiety derived from L-carnitine, or a derivative or analog thereof selected from Table 4.

80. The method of claim 67, wherein X is a moiety derived from metformin, or a derivative or analog thereof selected from Table 5.

81. The method of claim 67, wherein X is a moiety derived from berberine, or a derivative or analog thereof, and Z is a bile acid, or a derivative or analog thereof.

82. The method of claim 67, wherein X is a moiety derived from berberine, or a derivative or analog thereof, and Z is a fatty acid, or a derivative or analog thereof.

83. The method of claim 67, wherein X is a moiety derived from L-carnitine, or a derivative or analog thereof, and Z is a bile acid, or a derivative or analog thereof.

84. The method of claim 67, wherein X is a moiety derived from L-carnitine, or a derivative or analog thereof, and Z is a fatty acid, or a derivative or analog thereof.

85. The method of claim 67, wherein X is a moiety derived from metformin, or a derivative or analog thereof, and Z is a bile acid, or a derivative or analog thereof.

86. The method of claim 67, wherein X is a moiety derived from metformin, or a derivative or analog thereof, and Z is a fatty acid, or a derivative or analog thereof.

87. The method of claim 67, wherein Z is a moiety derived from ursodeoxycholic acid and X is a moiety derived from berberine.

88. The method of claim 67, wherein Z is a moiety derived from ursodeoxycholic acid and X is a moiety derived from L-carnitine.

89. The method of claim 67, wherein Z is a moiety derived from ursodeoxycholic acid and X is a moiety derived from one of metformin, coptisine, palmatine and jatrorrhizine.

90. The method of claim 67, wherein Z is moiety derived from ursodeoxycholic acid, or a derivative or analog, and X is a moiety derived from an unsaturated fatty acid.

91. The method of claim 90, wherein the unsaturated fatty acid is selected from Table 6.

92. The method of any of claims 77-91, wherein the linker comprises an amide bond.

93. The method of claim 92, wherein the linker comprises a moiety derived from an amino acid selected from Table 7.

94. The method of any of claims 77-91, wherein the linker comprises an ester bond.

95. The method of any of claims 67-94, wherein the pharmaceutical composition further comprises one or more of vitamin E, omega-3 fatty acids, S-adenosylmethionine, N-acetyl cysteine, silymarin, polyenylphosphatidylcholine, resveratrol, and vitamin D.
